(19) United States
(12) Patent Application Publication
(10) Pub. No.: US 2025/0387253 A1
(43) Pub. Date: Dec. 25, 2025

(54) SEMI-RIGID DORSO-LUMBAR BACK BRACES AND RELATED METHODS

(71) Applicant: DJO, LLC, Carlsbad, CA (US)

(72) Inventors: Constance QUINTY, Biarritz (FR); Luc THYS, Kortenberg (BE)

(21) Appl. No.: 19/306,140

(22) Filed: Aug. 21, 2025

Related U.S. Application Data
(63) Continuation of application No. PCT/US24/17840, filed on Feb. 29, 2024.
(60) Provisional application No. 63/450,583, filed on Mar. 7, 2023.

(51) Int. Cl. A61F 5/02

(52) U.S. Cl. CPC A61F 5/026

(57) ABSTRACT

A brace includes a bracing portion configured to be disposed against a portion of a back of a user and including a rigid or semi-rigid stay having a plurality of apertures with a same cross-section. The bracing portion includes at least first and second shoulder strap slots, each disposed in a respective top portion of the bracing portion, and at least first and second waist strap slots, each disposed in a respective bottom portion of the bracing portion. The brace includes a multi-strap harness configured to releasably couple with any one of the plurality of apertures of the stay and including at least first and second strap slots. The brace includes a plurality of straps, each coupled to a different slot of the bracing portion and/or the multi-strap harness to, thereby, wrap around a waist, a respective side, and/or shoulder of a user. Other braces and related methods are provided.

### CROSS-REFERENCE TO RELATED APPLICATIONS

This application is a bypass continuation of PCT Patent Application No. PCT/US2024/017840, filed Feb. 29, 2024, which claims priority to U.S. Provisional Patent Application No. 63/450,583, filed Mar. 7, 2023, the contents of all of which are each hereby incorporated by reference in their entirety.

### BACKGROUND

### Field of the Disclosure

The present disclosure relates generally to semi-rigid dorso-lumbar back braces and related methods.

### Description of the Related Technology

Back injuries and pathologies are common in the general population. Osteoporosis, vertebral fractures, kyphosis, acute and/or chronic back pain, osteolysis, muscular insufficiency, bad posture stemming from muscle weakness, hunched back, and juvenile Scheuermann's disease are just a few such back injuries and/or pathologies which may require stabilization of the dorso-lumbar region of the back of a sufferer.

However, current brace offerings can be difficult or cumbersome to adjust. Accordingly, there remains a need for improved semi-rigid dorso-lumbar back braces and related methods.

### SUMMARY

In some embodiments, a back brace configured for attachment to a user is provided. The brace includes a bracing portion configured to be disposed against a portion of a back of a user. The bracing portion includes a rigid or semi-rigid stay comprising a plurality of apertures, each having a same cross-section. The bracing portion includes at least a first shoulder strap slot and at least a second shoulder strap slot, each disposed in a respective top portion of the bracing portion. The bracing portion includes at least a first waist strap slot and at least a second waist strap slot, each disposed in a respective bottom portion of the bracing portion. The brace includes a multi-strap harness configured to releasably couple with any one of the plurality of apertures of the stay, the harness comprising a first strap slot and a second strap slot. The brace includes a plurality of straps, each coupled to a different slot of at least one of the bracing portion and the multi-strap harness to, thereby, wrap at least partly around a waist, a respective side and/or a respective shoulder of a user

A method of utilizing a back brace configured for attachment to a user is provided. The method includes releasably coupling a multi-strap harness into any one of a plurality of apertures of a rigid or semi-rigid stay embedded in a bracing portion of the brace. The method includes disposing the bracing portion of the brace against a portion of the back of the user. The method includes securing a waist strap around a waist of the user while the waist strap extends through slots in the bracing portion. The method includes securing a first strap to pass through a first shoulder strap slot of the bracing portion, around the front of the first shoulder of the user, diagonally back and down under the first underarm of the user, and through a first slot of the harness. The method includes securing a second strap to pass through a second shoulder strap slot of the bracing portion, around the front of the second shoulder of the user, diagonally back and down under the second underarm of the user, and through a second slot of the harness.

A method of manufacturing a back brace configured for attachment to a user is also provided. The method includes embedding a rigid or semi-rigid stay comprising a plurality of apertures, each having a same cross-section, within a bracing portion. The method includes forming at least a first shoulder strap slot and at least a second shoulder strap slot in respective top portions of the bracing portion. The method includes forming at least a first waist strap slot and at least a second waist strap slot in respective bottom portions of the bracing portion. The method includes providing a multi-strap harness configured to releasably couple with any one of the plurality of apertures of the stay, the harness comprising a first strap slot and a second strap slot. The method includes providing a plurality of straps, each configured to be coupled to, or through, a different slot of at least one of the bracing portion and the multi-strap harness for securing the brace against a back of a user.

### BRIEF DESCRIPTION OF THE DRAWINGS

FIG. 1 illustrates an embodiment of a bracing portion of a brace, according to some example embodiments;

FIG. 2 illustrates another embodiment of a bracing portion of a brace, according to some example embodiments;

FIGS. 3A, 3B and 3C each illustrate one of a small, medium and large sized bracing portion as shown in FIG. 2, according to some example embodiments;

FIG. 4 illustrates a perspective view of the bracing portion of FIG. 2, according to some example embodiments;

FIG. 5 illustrates another perspective view of the bracing portion of FIG. 2, according to some example embodiments;

FIG. 6 illustrates a side view of a metallic stay embedded within the bracing portion of any of FIGS. 1-5, according to some example embodiments;

FIG. 7 illustrates a front view of the metallic stay of FIG. 6;

FIG. 8 illustrates a perspective view of the metallic stay of FIGS. 6-7;

FIG. 9 illustrates a front view of the metallic stay of FIGS. 6-7 before shaping, according to some example embodiments;

FIG. 10 illustrates a side view of a portion of any of the bracing portions of FIGS. 1-5, according to some example embodiments;

FIG. 11 illustrates a side cutaway view of a portion of any of the bracing portions of FIGS. 1-5, according to some example embodiments;

FIG. 12 illustrates a back-facing side of a portion of the bracing portion of any of FIGS. 1-5 visibly comprising the metallic stay of FIGS. 6-9, according to some example embodiments;

FIG. 13 illustrates an upper portion of a back-facing side of the bracing portion of any of FIGS. 1-5 comprising back-facing padding, according to some example embodiments;

FIG. 14 illustrates a view of a side of the bracing portion opposite the back-facing side shown in FIG. 13, according to some example embodiments;

FIGS. 15A-15B illustrate a strap of a brace, according to some example embodiments;

FIG. 16 illustrates the strap of FIGS. 15A-15B laced through any of the bracing portions of FIGS. 1-5, according to some example embodiments;

FIG. 17 illustrates a first view of a strap harness of a brace, according to some example embodiments;

FIG. 18 illustrates a view of the strap harness opposite the view in FIG. 17, according to some example embodiments;

FIG. 19 illustrates the strap harness of FIGS. 17-18 being aligned with one of the apertures of the bracing portion of any of FIGS. 1-5, according to some example embodiments;

FIG. 20 illustrates a view of the back-facing side of any of the bracing portions of FIGS. 1-5 with the strap harness of FIGS. 17-18 inserted into apertures of the bracing portion, according to some example embodiments;

FIG. 21A illustrates a front view of a brace, according to some example embodiments;

FIG. 21B illustrates a back view of the brace of FIG. 21A;

FIG. 21C illustrates a backside view of a person wearing the brace of FIGS. 21A-21B;

FIG. 21D illustrates a backside view of a person wearing another brace, according to some example embodiments;

FIG. 22 illustrates a frontside view of the person wearing the brace of any of FIGS. 21A-21D, according to some example embodiments;

FIG. 23A illustrates a sternum strap of the brace of FIGS. 21A-22, according to some example embodiments;

FIG. 23B illustrates another sternum strap of the brace of FIGS. 21A-22, according to some example embodiments;

FIG. 24 illustrates a backside view of a person wearing another brace, according to some example embodiments;

FIG. 25 illustrates a side view of the person wearing the brace of FIG. 24, according to some example embodiments;

FIG. 26 illustrates a frontside view of the person wearing the brace of FIGS. 24-25, according to some example embodiments;

FIG. 27 illustrates a backside view of a brace utilizing another strap harness, according to some example embodiments;

FIG. 28 illustrates a cutaway view of a strap guide aligned with an aperture of the bracing portion of the brace of FIG. 27, according to some example embodiments;

FIG. 29 illustrates a backside view of a brace utilizing yet another strap harness, according to some example embodiments;

FIG. 30 illustrates a backside view of a person wearing the brace of any of FIGS. 27-29, according to some example embodiments;

FIG. 31 illustrates a side view of the person wearing the brace of FIGS. 27-29, according to some example embodiments;

FIG. 32 illustrates a frontside view of the person wearing the brace of FIGS. 27-29, according to some example embodiments;

FIG. 33 illustrates a flowchart of a method of utilizing a brace, in accordance with some embodiments;

FIG. 34 illustrates a flowchart of another method of utilizing a brace, in accordance with some embodiments; and

FIG. 35 illustrates a flowchart of a method of manufacturing a brace, in accordance with some embodiments.

### DETAILED DESCRIPTION

Embodiments of this disclosure relate to semi-rigid dorso-lumbar back braces and related methods. In some embodiments, such back braces and/or related methods may be indicated for conditions including, but not limited to, osteoporosis, consequences of stable vertebral fractures as a result of osteoporosis, kyphosis with chronic back pain, osteolysis, muscular insufficiency, bad posture of at least the thoracic spine as a result of muscle weakness, back pain, hunched back, and/or Juvenile Sheuermann's disease, among other conditions.

As will be described in greater detail below and as illustrated in the FIGs., embodiments of braces described herein are configured to provide support, stabilization and partial immobilization (e.g., restriction or control of movement) of the spine, to support stable spinal fractures, to relieve pain and facilitate correction of postural dysfunction from dorsal hyper-kyphosis and/or Parkinson's disease, to straighten and support the spine in the case of muscular insufficiency and/or weakness, and pain relief and/or support of spinal osteoarthritis.

First, a brief overview of the bones of the human spine for context is provided. At the very base of the spine is the coccyx. The coccyx is connected to the sacral vertebrae (from the coccyx, S5 to S1 in descending order up the spine). The S1 vertebra is connected to the lumbar vertebrae (from S1, L5-L1 in descending order up the spine). The L1 vertebra is connected to the thoracic vertebrae (from L1, T12-T1 in descending order up the spine). The T1 vertebra is connected to the cervical vertebrae (from T1, C7-C1 in descending order up the spine). And the skull is connected to C1. The spinal cord extends, from the brain, through the vertebrae, and each vertebra is separated from the adjacent vertebrae by respective discs. The cervical vertebrae are, generally, disposed in the neck, the thoracic vertebrae, generally, in the upper back, and the lumbar and sacral vertebrae, generally, in the lower back.

Braces disclosed herein are designed to relieve back pain, stabilize and support the lumbar and dorsal-lumbar spine (e.g., C7-S1) and, so, have a length that reaches up to at least the T6 vertebra of a wearer. Such braces are lightweight, making extended wear comfortable while maintaining design quality and durability, have slim and low-profile designs that fit under clothing, are breathable, anatomically shaped and pressure free, making them comfortable to wear, easy to remove, simple to adjust, yet rigid enough to keep shape for corrective action.

As will be described below, such braces may come in a plurality of different sizes, have adjustable strap lengths, differing stay lengths to accommodate different spine lengths, closures within hand reach, and/or allow for easy application of, for example, a backpack over the braces.

As will also be described below, portions of such braces may be constructed from high quality, innovative and attractive fabrics—e.g., hypoallergenic, PVC and latex free, washable, breathable, odor-free and/or moisture-wicking materials and/or fabrics for optimal thermoregulation and underlying air circulation. In some embodiments, unisex braces are offered in multiple colors, e.g., black or red.

While the present disclosure is not so limited, daily usage of such braces may exceed 12 hours, even where such braces are removed at night before sleep. Such braces are also suited for everyday activities (e.g., walking, sitting, lifting light objects), for stressful activities over the long-term, for indoor and/or outdoor use, and have device lifetimes that may exceed 12 months under such contexts.

Features of such braces that will be described in more detail below include but are not limited to a dorsal stay that is adjustable and/or moldable to fit the individual curvature of the patient and to keep the spine of the individual patient in a corrective position, hook and loop front closures that allow quick and easy adjustment, a pulling traction system having non-elastic straps and loops for targeted strap tension, personalized brace pressure, and substantially perfect lumbar stabilization.

Braces described herein provide an optimal fit at least in that a lumbar belt is configured to keep the abdominal area free for comfort, breathing, eating and/or moving.

Braces described herein provide optimal correction of kyphosis at least in part by designing the dorsal stay to include a plurality of apertures that each accommodate a different positioning for an adjustable strap guide to, thereby, achieve optimal strap orientation for achieving the effects and benefits of braces as described anywhere in this disclosure.

Braces described herein provide easy application at least in that a corrective strap comprises an anterior closure within hand reach, in that the brace is designed to provide easy first-step over-the-shoulders application, and in that the brace comprises differential strap coloring to provide easy differentiation between the different straps of the brace.

Braces described herein provide ease of adjustability at least in that strap lengths are adjustable, in that the pre-bent dorsal stay within the brace imitates the anatomical curvature of the spine of the particular user, and in that the plurality of apertures in the dorsal stay improves both breathability and provides another dimension of strap adjustability.

Braces described herein provide increased wearing comfort at least in that an additional, removable anterior thoracic strap may be provided to relieve axillar pressure, in that the dorsal stay has a central zone pre-shaped to avoid pressure points, and in that the brace has a slim design specifically configured to easily fit underneath clothing.

Braces described herein also allow for less inventory at least in that a select number of sizes (e.g., 3) allow proper fitting with a broad variety of body types and sizes.

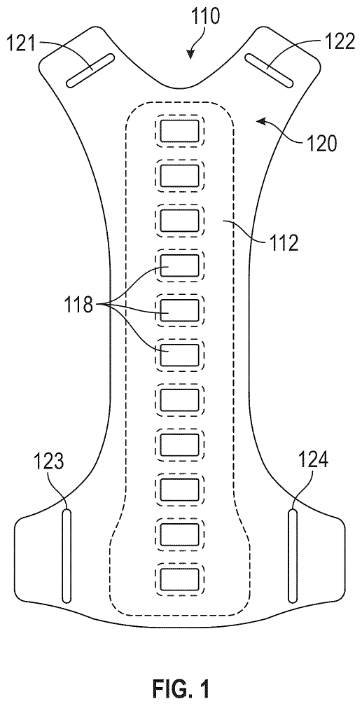

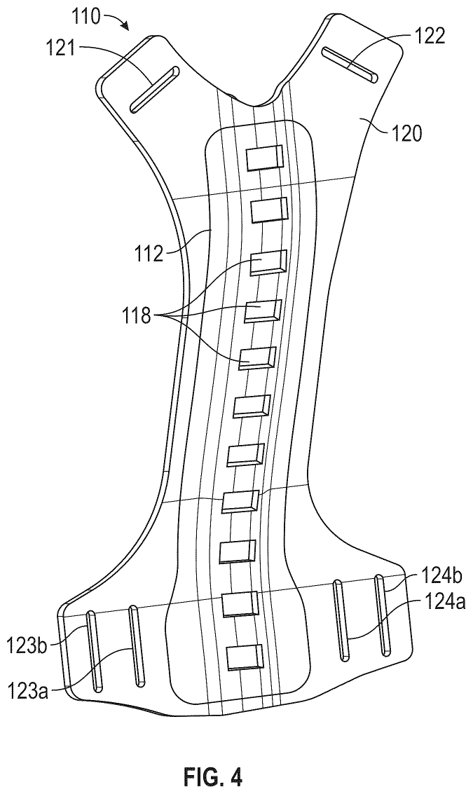

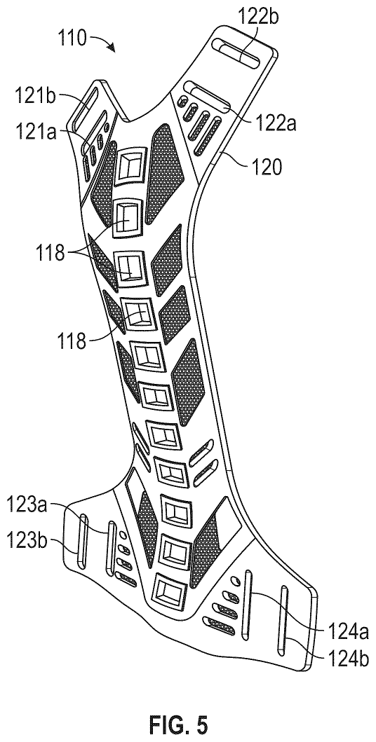

FIGS. 1-5 each illustrate a different view of an embodiment of a bracing portion 110 of a brace 100, according to some example embodiments. In some embodiments, bracing portion 110 comprises a single left shoulder strap slot 121 and a single right shoulder strap slot 122 (see, e.g., FIGS. 1-4). In some other embodiments, bracing portion 110 comprises double left shoulder strap slots 121a, 121b and double right shoulder strap slots 122a, 122b (see, e.g., FIG. 5). In all such embodiments, the shoulder strap slots are configured for coupling shoulder straps.

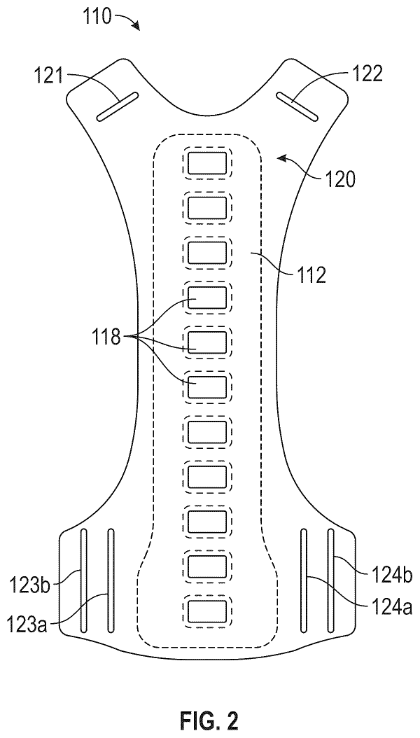

Further, in some such embodiments, bracing portion 110 comprises a single left waist strap slot 123 and a single right waist strap slot 124 (see, e.g., FIG. 1). In some other such embodiments, bracing portion 110 comprises double left waist strap slots 123a, 123b and double right waist strap slots 124a, 124b (see, e.g., FIGS. 2-5). In all such embodiments, the waist strap slots are configured for coupling one or more waist strap(s).

Bracing portion 110 is configured to be disposed against a portion of a back of a user along substantially an entire length of bracing portion 110 to stabilize and support the lumbar and dorso-lumbar spine, specifically, at least vertebrae T1-S1. Accordingly, bracing portion 110 may be adjustable, moldable, and/or otherwise morpho-adaptable to fit the individual curvature of the pertinent portion of the back of the individual patient and, thereby, keep the spine in a corrective position. To aid in providing such corrective support, bracing portion 110 comprises a metallic stay 112 sandwiched between and/or embedded within a plurality of material layers 120 that, in the aggregate, form bracing portion 110.

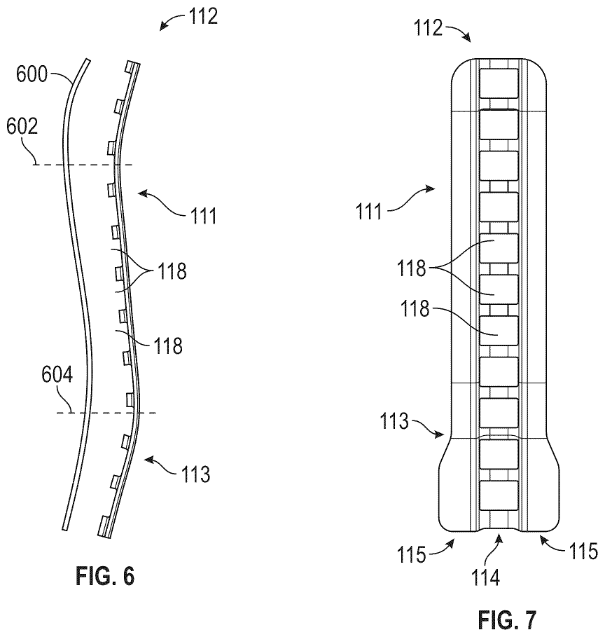

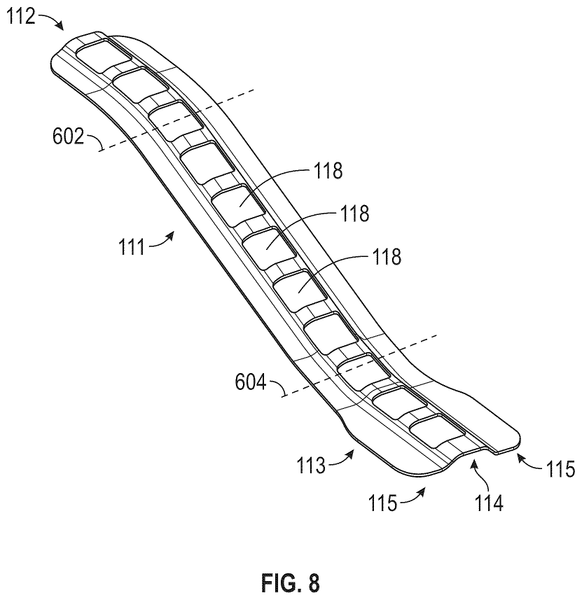

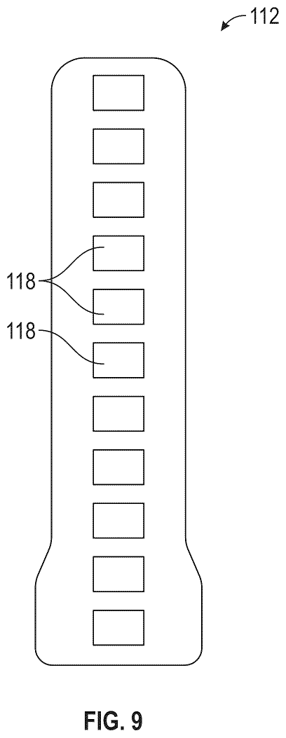

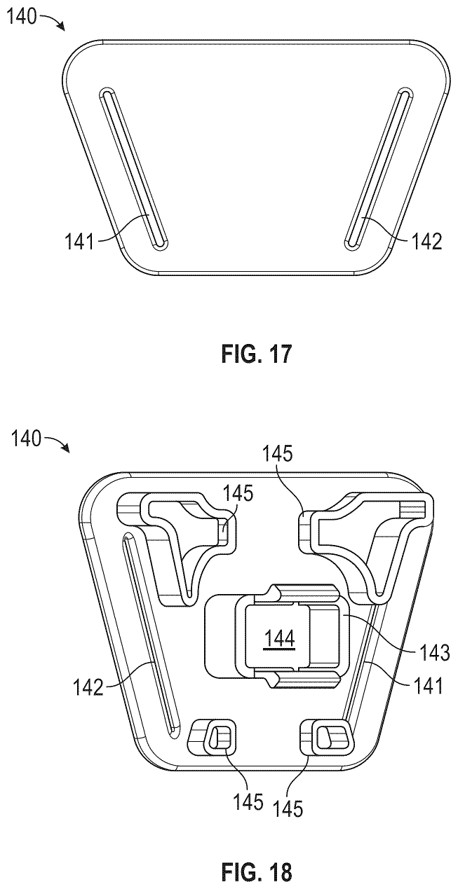

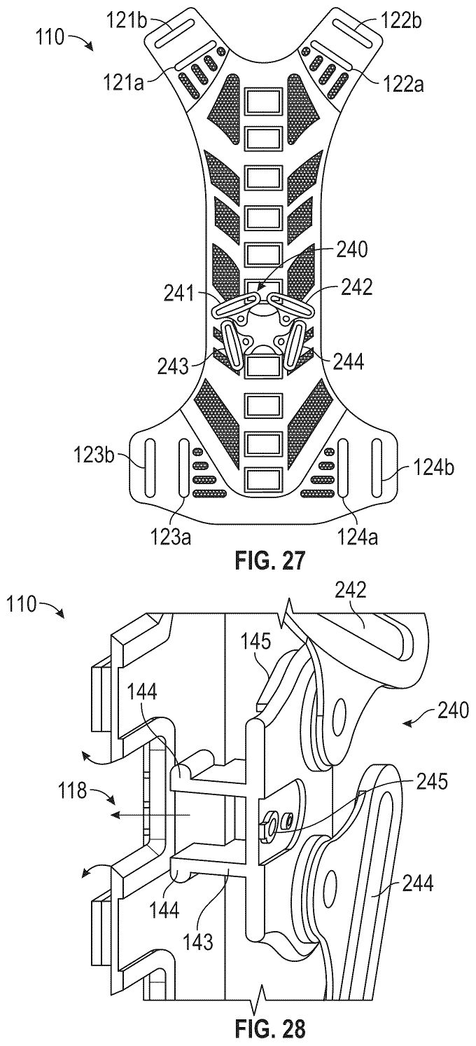

FIGS. 6-9 each illustrate a different view of metallic stay 112, according to some example embodiments. Metallic stay 112 comprises a plurality of apertures 118, each configured to receive and, thereby, secure a multi-strap harness 140, 240, 340, for example, as described in connection with at least FIGS. 17-19 and 27-29. Contributing to its semi-rigid, supportive configuration, metallic stay 112 may be formed of a semirigid and/or malleable material, such as aluminum and may be stamped, bent, formed and/or molded to have one or more curvatures in one or more orthogonal planes, as can be seen for example, in FIGS. 6-8. Accordingly, metallic stay 112 may have any suitable thickness, e.g., 2 mm. FIG. 6 illustrates a side view of metallic stay 112, while FIG. 7 illustrates a straight-on view of metallic stay 112, 90 degrees rotated from the view of FIG. 6. And FIG. 8 illustrates a perspective view of metallic stay 112.

FIG. 6 illustrates a dorsal curvature 600 of the back of a specific user of brace 100. Specifically, dorsal curvature 600 has a substantially “S”-shape and comprises a first inflection point 602 and a second inflection point 604. As viewed from the back-facing side, curvature 600 is concave from the top of curvature 600 to a point substantially midway between first inflection point 602 and second inflection point 604. As viewed from the back-facing side, from the point substantially midway between first inflection point 602 and second inflection point to the bottom of curvature 600, curvature 600 is convex.

As shown in at least FIG. 6, metallic stay 112 is bent, molded or otherwise formed to mimic and/or track curvature 600 (e.g., in the sagittal plane) such that, when brace portion 110, comprising stay 112 therein, is properly disposed against the back of the user, properly formed, molded and/or bent stay 112 comprises inflection points matching (e.g., disposed immediately adjacent to) inflection points 602, 604 of the back curvature 600 of the particular user. Accordingly, stay 112 also has a substantially “S”-shape and an upper portion 111 having a back-facing concave curvature that extends between a top of stay 112 and a point substantially midway between the inflection points of stay 112 corresponding to inflection points 602, 604 of back curvature 600. Stay 112 also has a lower portion 113 having a back-facing convex curvature that extends between a bottom of stay 112 and the point substantially midway between the inflection points of stay 112 corresponding to inflection points 602, 604 of back curvature 600.

Accordingly, upper portion 111 of metallic stay 112 substantially mimics the dorsal curvature of the lower thoracic portion of the back of the user (e.g., T6-T12), and lower portion 113 of metallic stay 112 substantially mimics the dorsal curvature of the lumbar portion of the back of the user (e.g., L1-L5). In this way, pressure may be distributed evenly, or more evenly, on the user's back, thereby making brace 100 more comfortable.

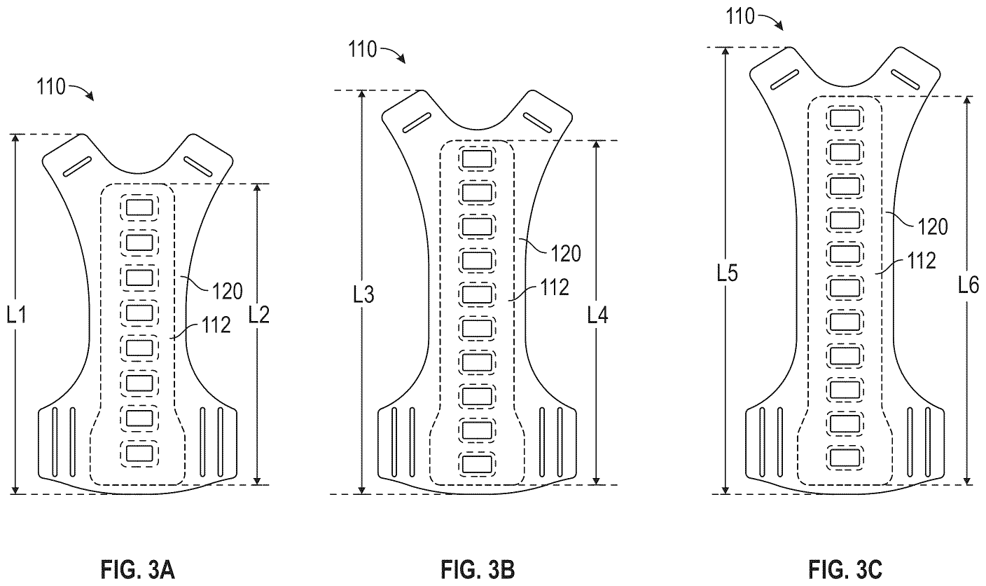

In some embodiments, the exact dimensions of metallic stay 112 and, so, of upper and lower portions 111, 113 may vary according to the size of brace 100 and/or of bracing portion 110. In some embodiments, a “small” size for brace 100 may correspond to a S1-C7 vertebral extent range of 42-52 cm, may utilize a top-to-bottom length L₂ of 41 cm for metallic stay 112, and a top-to-bottom length L₁ of 49 cm for bracing portion 110 for example as shown in FIG. 3A. A “medium” size for brace 100 may correspond to a S1-C7 vertebral extent range of 52-62 cm, may utilize a top-to-bottom length L₄ of 47 cm for metallic stay 112, and a top-to-bottom length L₃ of 55 cm for bracing portion 110 for example as shown in FIG. 3B. And a “large” size for brace 100 may correspond to a S1-C7 vertebral extent range of 62-72 cm, may utilize a top-to-bottom length L₆ of 53 cm for metallic stay 112, and a top-to-bottom length L₅ of 61 cm for bracing portion 110 for example as shown in FIG. 3C. As brace 100 is at least minimally adjustable, the dorsal curvature of at least lower portion 113 of metallic stay 112 may be adjusted by a practitioner to more exactly conform to the specific dorso-lumbar region of the back of the specific user of brace 100.

As shown in FIG. 7, metallic stay 112 may also have a variable width. For example, upper portion 111 may have a first width, while lower portion 113 may flare from the first width to a second width greater than the first width. In some embodiments, metallic stay 112 may also comprise rounded corners having a radius of curvature large enough to prevent metallic stay 112, and/or bracing portion 110 housing metallic stay 112, from digging to the back of the user.

As best shown in FIG. 8, metallic stay 112 also has a curvature in the transverse plane. For example, a back-facing side of a central longitudinal portion 114, running from top to bottom of metallic stay 112, may have a concave curvature such that central portion 114 extends away from the back of the wearer and away from lateral longitudinal portions 115 that extend along each side of central portion 114 from top to bottom of metallic stay 112. The concave curvature of central portion 114 provides space along the entire length of the back-facing side of metallic stay 112 for the backbone and spine of the user.

As shown in FIG. 7, apertures 118 are disposed within central portion 114 of metallic stay 112. In some embodiments, the exact dimensions of metallic stay 112 and, so, of central 114 and lateral portions 115, may vary according to the size of brace 100 and/or of bracing portion 110. Together, the dorsal, back-facing concave curvature of upper 111, the dorsal, back-facing convex curvature lower 113 portions, and the back-facing concave curvature of central 114 portion, between lateral portions 115 of metallic stay 112, allow for drastic improvements in both support and comfort for the patient.

As stated above, bracing portion 110 comprises a metallic stay 112 sandwiched between and/or embedded within a plurality of material layers 120 that, in the aggregate, form bracing portion 110. Discussion now turns to these material layers.

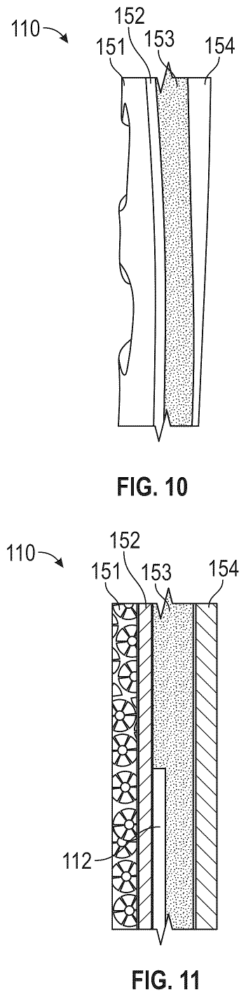

FIG. 10 illustrates a side view of a portion of bracing portion 110, according to some example embodiments, while FIG. 11 illustrates a cutaway view of this same portion, showing each of the material layers forming bracing portion 110. In some embodiments, bracing portion 110 comprises 5 separate layers or components, though the present disclosure is not so limited. An outer layer 151 may comprise an injected polyurethane (e.g., a Nanoflash process), for example to enhance cleaning and durability of bracing portion 110 compared to conventional materials. Outer layer 151 may be disposed and/or coupled directly against a textile layer 152 (e.g., an Ozone textile), for example to enhance breathability of bracing portion 110 compared to conventional materials. In some embodiments, metallic stay 112 may be sandwiched between textile layer 152 and a padding layer 153 (e.g., 6 mm laminated foam), for example to enhance wearing comfort compared to conventional materials. Accordingly, some portions of textile layer 152 may be disposed and/or coupled directly against metallic stay 112 while some other portions of textile layer 152 may be disposed and/or coupled directly against padding layer 153. Likewise, some portions of padding layer 153 may be disposed and/or coupled directly against metallic stay 112 while some other portions of padding layer 153 may be disposed and/or coupled directly against textile layer 152. Padding layer 153 may be disposed and/or coupled directly against an absorbent inner textile layer 154 (e.g., an absorbent, perforated, antibacterial Onsteam fabric), for example to enhance comfort and breathability compared to conventional materials. Material layers 120 may be adhered or otherwise bonded to each other utilizing any suitable method, for example, adhesive.

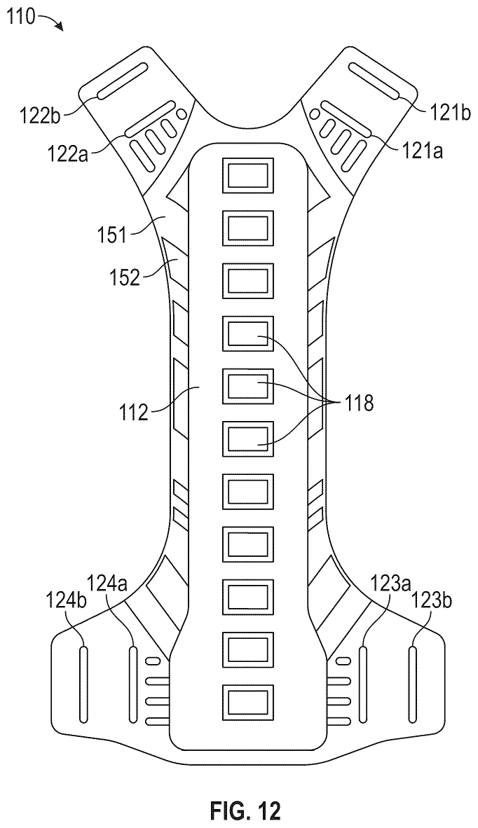

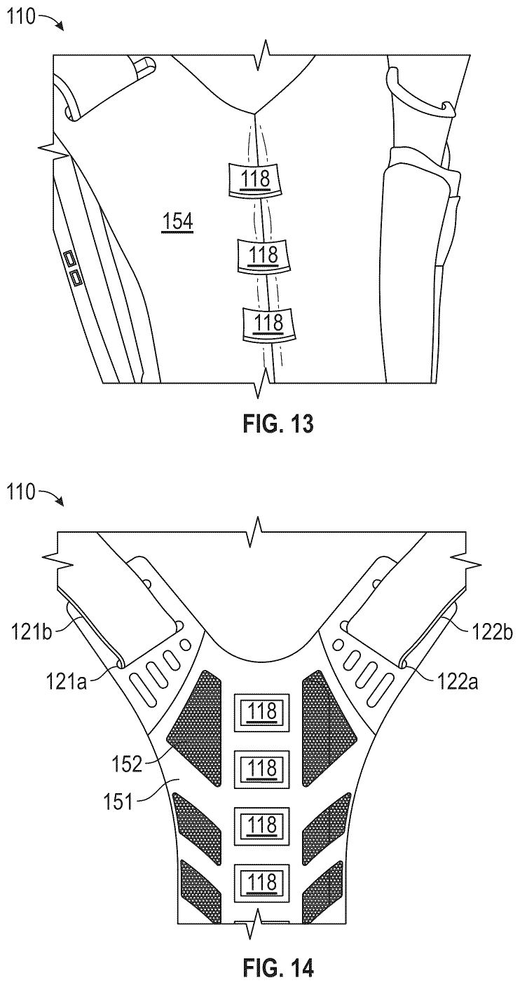

FIG. 12 illustrates metallic stay 112 disposed on textile layer 152, with portions of outer layer 151 (disposed on an opposite side of textile layer 152) visible, through textile layer 152, as the darkened patterned portions on textile layer 152. FIG. 13 provides a view of the back-facing side (e.g., inner textile layer 154) of an upper portion of bracing portion 110, while FIG. 14 provides a view opposite that of FIG. 13, showing outer layer 151 and textile layer 152 thereunder. FIGS. 12-14 also illustrates how the plurality of apertures 118 of metallic stay and any of slots 121a-b, 122a-b, 123a-b, and 124a-b present are also cut out of each layer forming bracing portion 110.

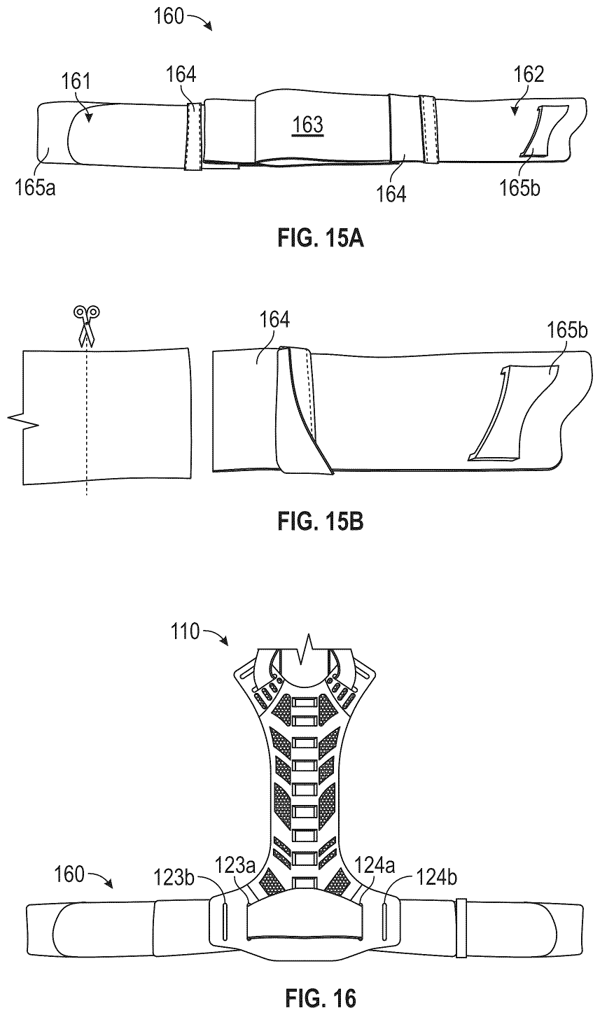

FIGS. 15A-15B illustrate a waist strap 160 for brace 100, according to some example embodiments. Waist strap 160 comprises an inner front closure 161, an outer front closure 162 and a central strap portion 163. In some embodiments, inner front closure 161, outer front closure 162 and central strap portion 163 all have a same width (e.g., 9 cm). However, the present disclosure is not so limited. Inner front closure 161 is fixedly coupled to one end of central strap portion 163. Outer front closure 162 is coupled to the other end of central strap portion 163 via a crocodile clip 164. For example, the mating end of central strap portion 163 may comprise one or both of a hook or loop material (e.g., velvet) and an inner surface of crocodile clip 164 may comprise the other or both of the hook or loop material such that, when disposed between opposing flaps of crocodile clip 164, the mating end of central strap portion 163 is adjustably fixed to the inner surfaces of crocodile clip 164. Such use of crocodile clip 164 increases a surface area of hook and loop material disposed on the crocodile clip 164 and mating surfaces of central strap portion 163 and, thereby, increases an amount tension that waist strap 160 is capable of supporting. In some embodiments, the mating end of central strap portion 163 may be cut before coupling to crocodile clips 164, so as to provide central strap portion 163 with a desired length and/or width to accommodate a wide variety of waist sizes and shapes of specific users. In alternative embodiments, both inner front closure 161 and outer front closure 162 are configured to be coupled to central strap portion 163 via a respective crocodile clip 164, which may further improve the adjustability of waist strap 160. In yet other embodiments, inner front closure 161 may be adjustably fixable to its end of central strap portion 163, while outer front closure 162 is sewn or welded to central strap portion 163.

In some embodiments, inner front closure 161 may comprise a loop or pocket 165a configured to receive fingers of a wearer while waist strap 160 is being tightened around the waist of a user, thereby providing a feature with which to grasp one side of strap 160 more tightly and that allows the user to provide an increased amount of tension in strap 160 during fixation. In some embodiments, outer front closure 162 may comprise a loop or pocket 165b configured to receive fingers of a wearer while waist strap 160 is being tightened around the waist of a user, thereby providing a feature with which to grasp the other end of strap 160 more tightly and that allows the user to provide an increased amount of tension in strap 160 during fixation.

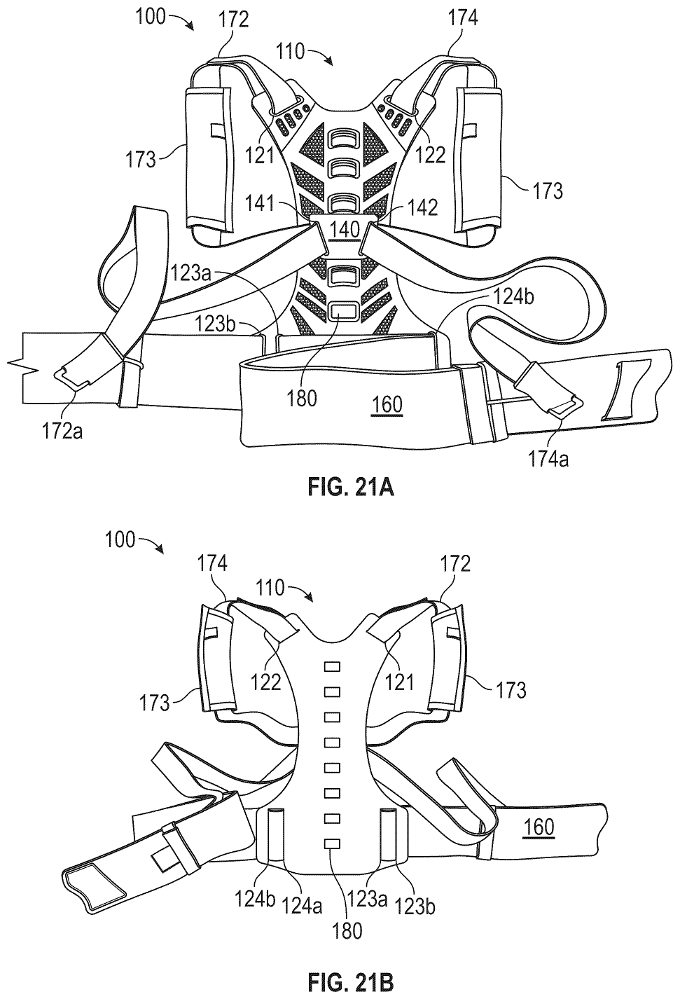

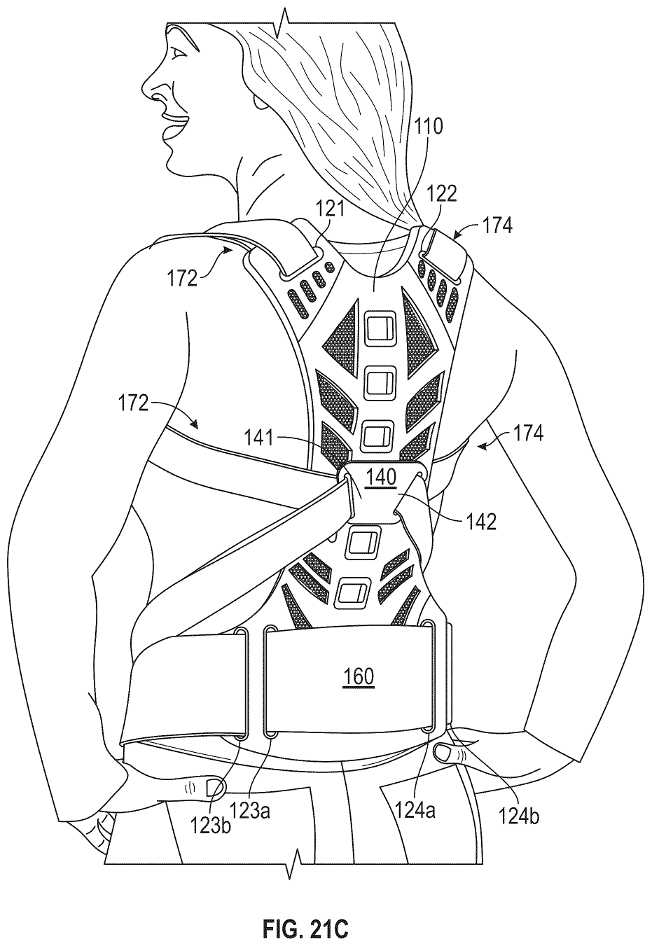

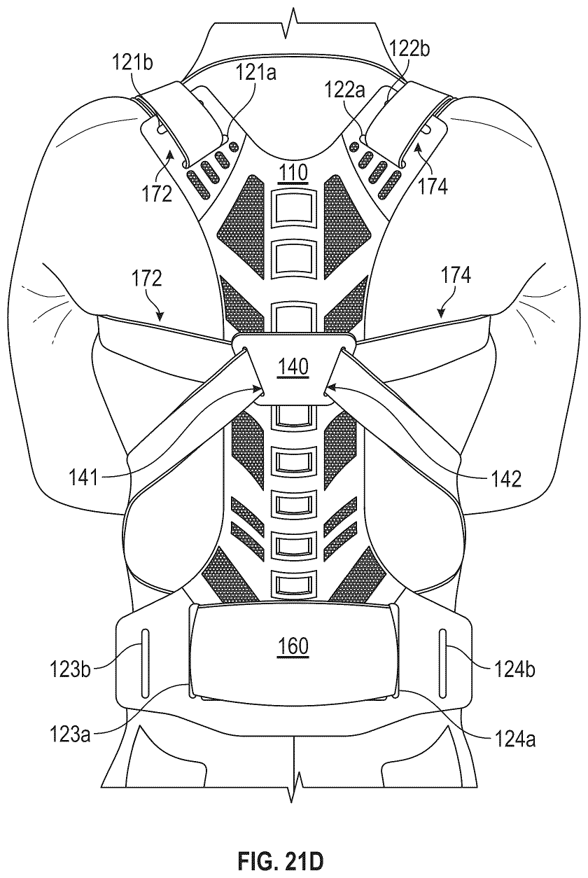

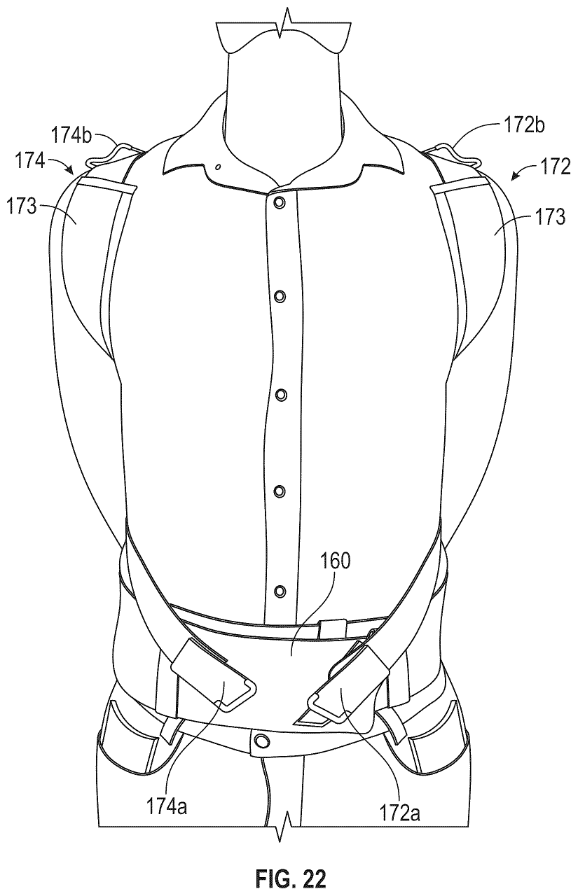

As shown in FIG. 16, waist strap 160 may be fed through at least one left waist strap slot (e.g., 123 where brace portion 110 comprises a single left waist strap slot, 123a and/or 123b where brace portion 110 comprises dual left waist strap slots) and at least one right waist strap slot (e.g., 124 where brace portion 110 comprises a single right waist strap slot, 124a and/or 124b where brace portion 110 comprises dual right waist strap slots) (see, e.g., FIG. 21D). In some embodiments where brace portion 110 comprises dual right waist strap slots, waist strap 160 may be fed through both left waist strap slots (e.g., 123a, 123b) and through both right waist strap slots (e.g., 124a, 124b) (see, e.g., FIG. 21C). When strap 160 is secured through bracing portion 110 and around the waist of a user, strap 160 effectively secures a bottom portion of bracing portion 110 snuggly, but comfortably, around the waist of the user.

Back and/or shoulder straps may also be coupled to bracing portion 110 utilizing a multi-strap harness 140 that is, itself, configured to be secured within any one of the plurality of apertures 118 in bracing portion 110 based on the size of the user and the bracing effect desired. Harness 140 is illustrated as having a substantially trapezoidal shape, for example, having a wider horizontal top edge compared to a relatively narrower horizontal bottom edge, and side edges that symmetrically slope between the top and bottom edges. A first strap slot 141 is disposed parallel to one of the side edges and a second strap slot 142 is disposed parallel to the other of the side edges. In some embodiments, as shown in FIG. 17, an outward facing surface of harness 140 is substantially flat.

As illustrated in FIG. 18, a back-facing side of harness 140 may comprise an extension 143 having one or more snap clips 144 configured to interface with, clip into, or interference fit with, any one of the plurality of apertures 118 in metallic stay 112 and bracing portion 110.

In some embodiments, the back-facing side of harness 140 may also comprise one or more spacers 145 configured to contact an outward-facing surface of bracing portion 110 when harness 140 is properly secured within one of apertures 118, thereby stabilizing harness 140 against rotation and/or lateral shifting within aperture 118. In some embodiments, harness 140 comprises a first spacer 145 disposed immediately adjacent a bottom of slot 141 and a second spacer 145 disposed immediately adjacent a bottom of slot 142. Similarly, in some embodiments, harness 140 comprises a third spacer 145 disposed immediately adjacent a top of slot 141 and a fourth spacer 145 disposed immediately adjacent a top of slot 142. In some such embodiments, third and fourth spacers 145 have a larger, wider form factor compared to first and second spacers 145, as third and fourth spacers 145 are disposed nearer the top, wider edge of harness 140 and first and second spacers 145 are disposed nearer the bottom, narrower edge.

When straps are properly fed through harness 140, and harness 140 is properly secured within one of apertures 180, harness 140 is configured to optimize the application of corrective traction to the back and/or shoulders of the user of brace 100.

As can be seen in FIG. 18, one or more of spacers 145 may have a first height to a lateral side of spacer(s) 145 and may slope to a second heigh that is less than the first height toward a medial side of spacer(s) 145. This sloping from a full first height at the lateral side to a reduced second height at the medial side of spacer(s) 145 may allow spacer(s) 145 to mate against portions of bracing portion 110 along an entire perimeter of apertures 118 when harness 140 is properly clipped and disposed within the aperture 118. This aspect forms a complementary shape to the portions of bracing portion 110 shaped by the curvature of central portion 114 of stay 112 extending away from lateral portions 115 of stay 112.

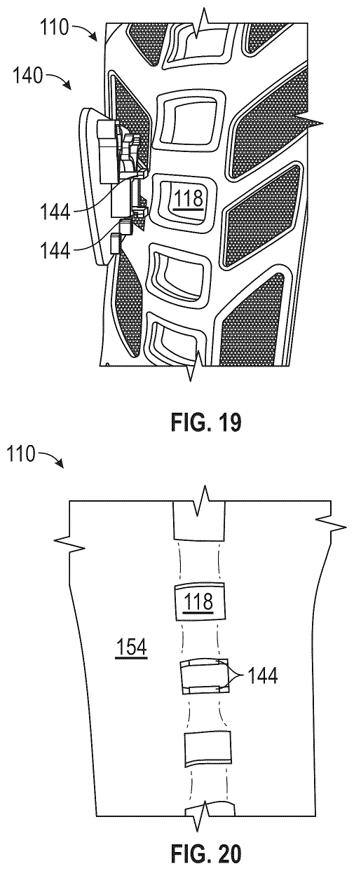

FIG. 19 illustrates harness 140 in the process of alignment with one of apertures 118 of bracing portion 110, while FIG. 20 illustrates harness 140 inserted into an aperture 118 and snap clips 144 snapped into and/or around sidewalls of the aperture 118 as viewed from the back-facing side of bracing portion 110.

FIG. 21A illustrates a front view of brace 100, according to some example embodiments, while FIG. 21B illustrates a back view of brace 100 shown in FIG. 21A. FIG. 21C illustrates a rear view of a person wearing brace 100 shown in FIGS. 21A and 21B, while harness 140 is engaged in an appropriate aperture 118 of bracing portion 110. FIG. 21D illustrates a rear view of a person wearing a slightly different version of brace 100 while harness 140 is engaged in an appropriate aperture 118 of bracing portion 110. FIG. 22 illustrates a front view of the person of either of FIG. 21C or 21D wearing the respective brace 100. As illustrated, first strap 172 is configured to be fed through first slot 141. A first portion of first strap 172 extends diagonally down from first slot 141 and around a side of the user, with a corresponding first end 172a of first strap 172 configured to be adjustably coupled to an anterior portion of secured waist strap 160. Accordingly, first end 172a of first strap 172 and waist strap 160 may each comprise hook and/or loop fasteners. In some embodiments, for example as shown in FIGS. 21A-21C, first end 172a of first strap 172 may comprise a ring or other grasping feature configured for the user to grab ahold of (e.g., with a finger disposed through the ring) to provide increased tension when adjusting and setting first strap 172. A second portion of first strap 172 extends diagonally up from first slot 141, around an underarm of the user, and up and around a front shoulder of the user, with a corresponding second end 172b of first strap 172 configured to be threaded through at least one shoulder strap slot in bracing portion 110 (e.g., slot 121a and/or 121b in FIG. 21D, slot 121 in FIG. 21C). Accordingly, second end 172b of first strap 172 may comprise hook and/or loop fasteners, which allow second end 172b of first strap 172 to extend though slot 121, 121a, 121b double back, and secure to itself. In some embodiments, second end 172b of first strap 172 may additionally (or alternatively) comprise a button snap so as to allow second end 172b of first strap 172 to loop through slot 121, 121a, and/or 121b and snap onto itself.

In some embodiments, as best illustrated in FIG. 22, the second portion of first strap 172 extends through an underarm pad 173, which is configured to increase comfort at the underarm. Accordingly, first strap 172 passes through a slot (e.g., 121a and/or 121b in FIG. 21D, 121 in FIG. 21C), around the front of the shoulder, diagonally back and down under the underarm, through first slot 141 of harness 140, diagonally down around the side of the user to couple to an anterior of waist strap 160. In this way, since fixation for first strap 172 is positioned anteriorly, and within hand reach of the user, a degree of posterior retraction of the user's shoulders may be adjusted according to patient needs during daily activities. While not shown in detail in FIGS. 21A-22, first end 172a and/or second end 172b of first strap 172 may comprise crocodile clips similar to those described in connection with waist strap 160. Accordingly, a length of first strap 172 may be adjusted in a similar way to that described for waist strap 160.

As illustrated, second strap 174 is configured to be fed through second slot 142. A first portion of second strap 174 extends diagonally down from second slot 142 and around a side of the user, with a corresponding first end 174a of second strap 174 configured to be adjustably coupled to an anterior portion of secured waist strap 160. Accordingly, first end 174a of second strap 174 and waist strap 160 may each comprise hook and/or loop fasteners. In some embodiments, for example as shown in FIGS. 21A-21C, first end 174a of second strap 174 may comprise a ring or other grasping feature configured for the user to grab ahold of (e.g., with a finger disposed through the ring) to provide increased tension when adjusting and setting second strap 174. A second portion of second strap 174 extends diagonally up from second slot 142, around an underarm of the user, and up and around a front shoulder of the user, with a corresponding second end 174b of second strap 174 configured to be threaded through at least one shoulder strap slot in bracing portion 110 (e.g., slot 122a and/or 122b in FIG. 21D, slot 122 in FIG. 21C). Accordingly, second end 174b of second strap 174 may comprise hook and/or loop fasteners, which allow second end 174b of second strap 174 to extend though slot 122, 122a, 122b double back, and secure to itself. In some embodiments, second end 174b of second strap 174 may additionally (or alternatively) comprise a button snap so as to allow second end 174b of second strap 174 to loop through slot 122, 122a, and/or 122b and snap onto itself.

In some embodiments, as best illustrated in FIG. 22, the second portion of second strap 174 extends through an underarm pad 173, which is configured to increase comfort at the underarm. Accordingly, second strap 174 passes through a slot (e.g., 122a and/or 122b in FIG. 21D, 122 in FIG. 21C), around the front of the shoulder, diagonally back and down under the underarm, through second slot 142 of harness 140, diagonally down around the side of the user to couple to an anterior of waist strap 160. In this way, since fixation for second strap 174 is positioned anteriorly, and within hand reach of the user, a degree of posterior retraction of the user's shoulders may be adjusted according to patient needs during daily activities. While not shown in detail in FIGS. 21A-22, first end 174a and/or second end 174b of second strap 174 may comprise crocodile clips similar to those described in connection with waist strap 160. Accordingly, a length of second strap 174 may be adjusted in a similar way to that described for waist strap 160.

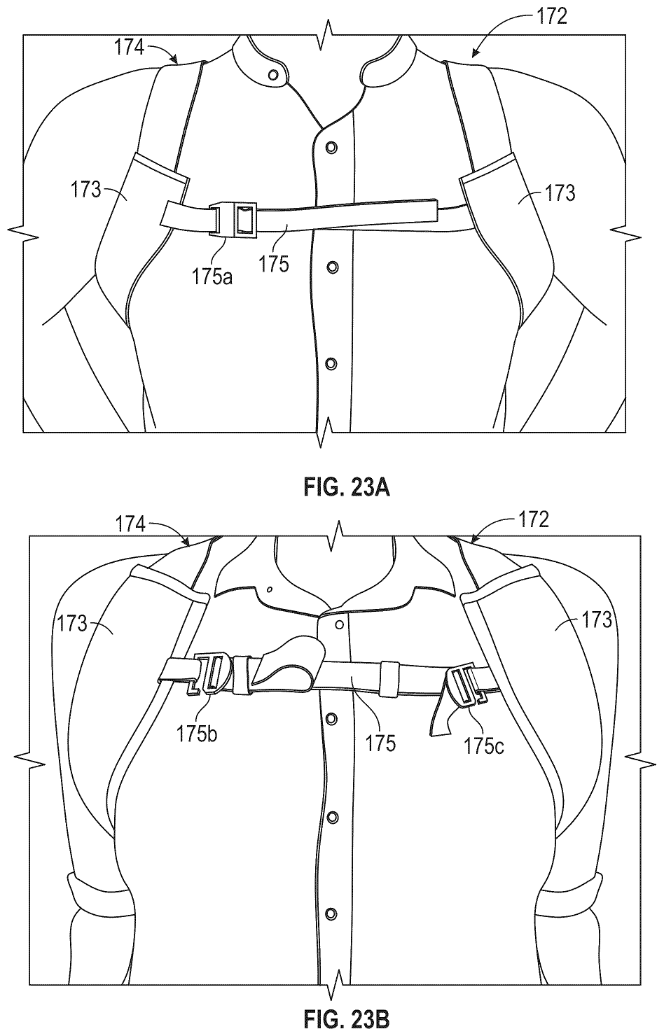

According to some embodiments, FIGS. 23A and 23B each illustrate a sternum strap 175 releasably coupled between underarm pads 173 of each of straps 172, 174. Sternum strap 175, coupled at each end to respective underarm pads 173, pulls underarm pads 173 medially away from the underarm, thereby relieving pressure under the armpits of the user that would otherwise be caused by straps 172, 174. In this way, sternum strap 175 provides auxiliary support for user comfort.

In the embodiment shown by FIG. 23A, sternum strap 175 comprises a first portion coupled to one underarm pad 173, a second portion coupled to the other underarm pad 173 and a buckle 175a coupling the first and second portions. Sternum strap 175 may be tightened by pulling on an end of one of the first and second portions that is doubled back through buckle 175a.

In the embodiment shown by FIG. 23B, sternum strap 175 may comprise a first buckle 175b, a first loop portion coupled to one underarm pad 173 and releasably coupled to the first loop portion, a second buckle 175c, a second loop portion coupled to the other underarm pad 173 and releasably coupled to the second loop portion, and a third portion coupled between the first and second buckles 175b, 175c. Sternum strap 175 may be tightened by pulling on the end of third portion that is doubled back through first buckle 175b or by pulling on the end of third portion that is doubled back through second buckle 175c.

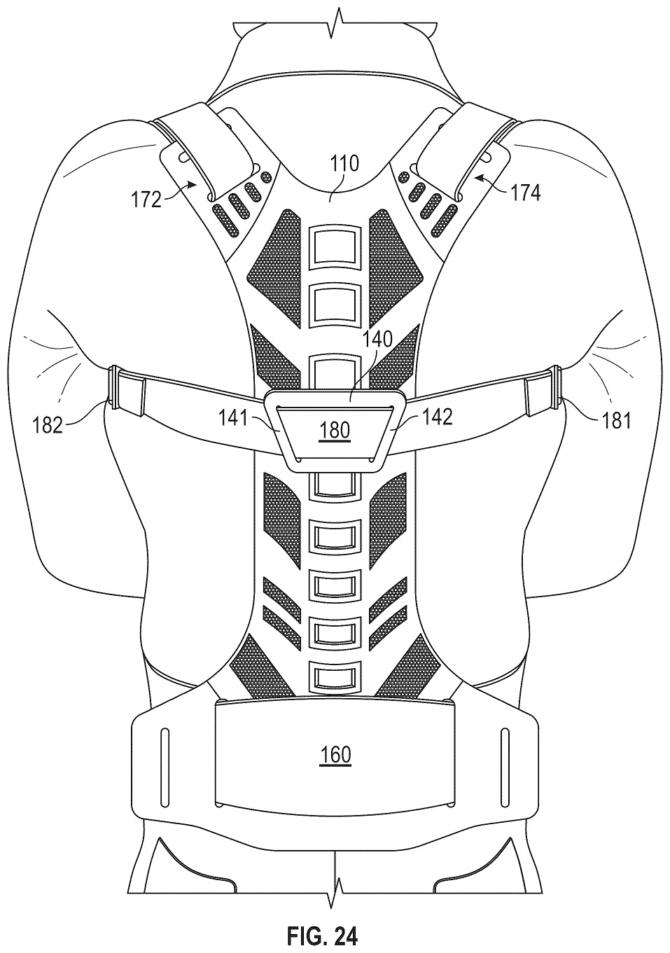

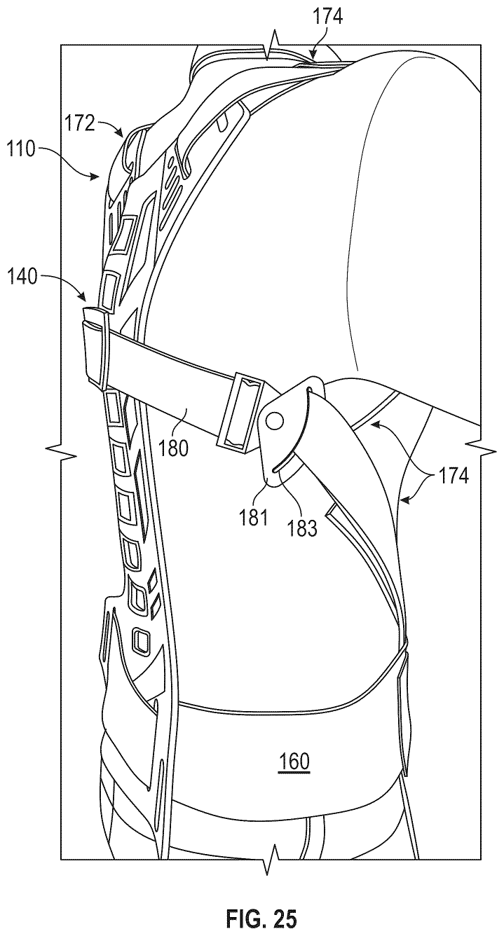

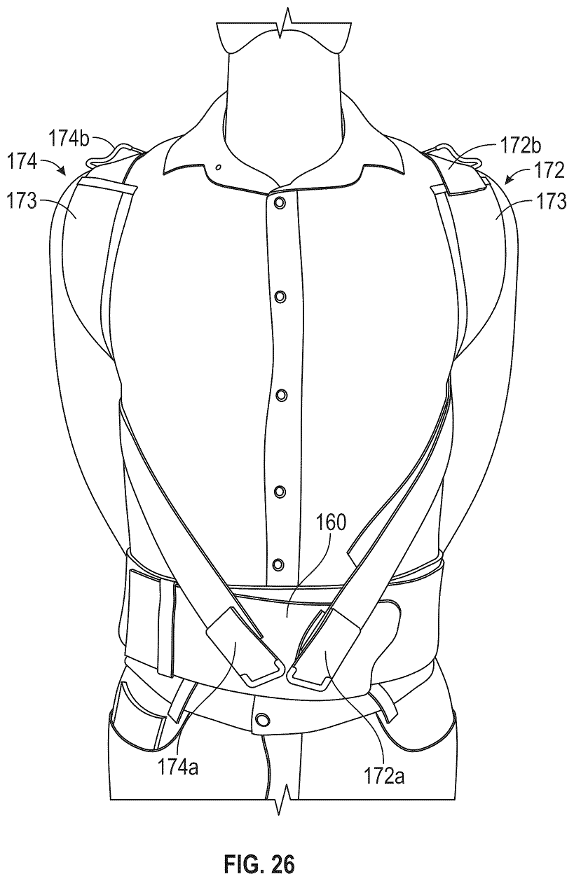

FIGS. 24-26 illustrates different views of a person wearing brace 100, however, brace 100 further utilizing an elastic cross strap 180 having end buckles 181, 182 to indirectly secure first and second straps 172, 174 to harness 140, rather than first strap 172 being directly fed through slot 141 and second strap 174 being directly fed through slot 142 of harness 140. For example, in embodiments according to FIGS. 24-26, cross strap 180 is configured to be fed through first and second slots 141, 142 of harness 140. A first end of cross strap 180 comprises a first buckle 181 having a first buckle slot 183, while a second end of cross strap 180 comprises a second buckle 182 having a second buckle slot 184 (not visible in FIGS. 24-26 but identical to slot 183). In some embodiments, each of buckles 181, 182 comprises a rivet such that a portion of the buckle in which the respective slot 183, 184 is disposed is configured to pivot with respect to another portion of the buckle to which one end of cross strap 180 is coupled (slot 184 not visible in the FIGS. 24-26, but see slot 183 of buckle 181 in FIG. 25). In some embodiments, each of buckles 181, 182 are bi-injection, comprising a rigid buckle as well as a soft inner surface configured to be disposed against the body of the user. In operation, first strap 172 may interact with all portions of brace 110 as previously described in connection with at least FIGS. 21A-23 except that, instead of first and second straps passing directly through first and second slots 141, 142 in harness 140, first and second straps each pass directly through first and second slots 183, 184 in first and second buckles 181, 182, and cross strap 180, itself, passes directly through first and second slots 141, 142 in harness 140. In this way, straps 172, 174 do not extend as far posteriorly as they otherwise would, for example, in arrangements as shown in FIGS. 21-23 and, therefore, straps 172, 174 may cut into the underarm of the user less when cross strap 180 and buckles 181, 182 are utilized.

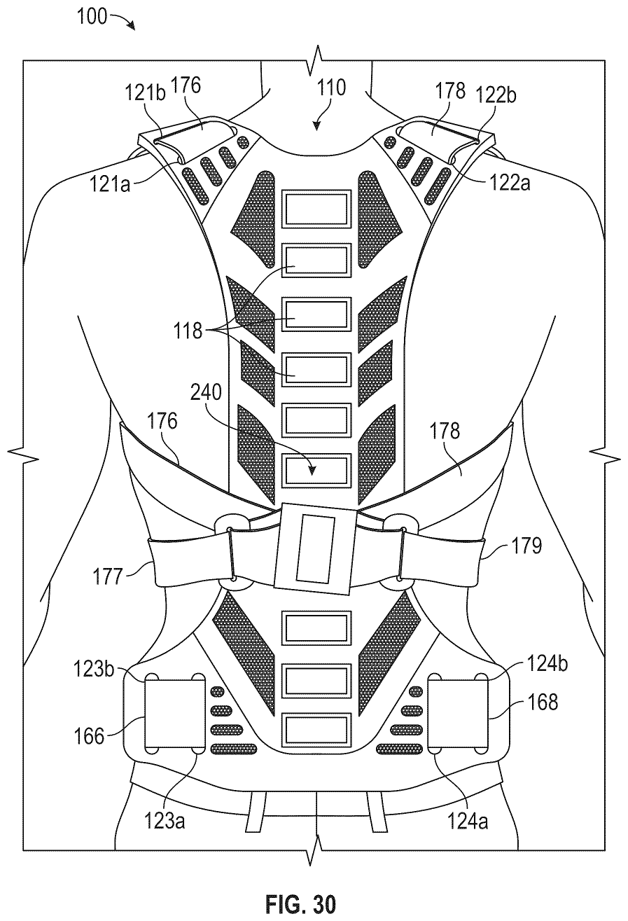

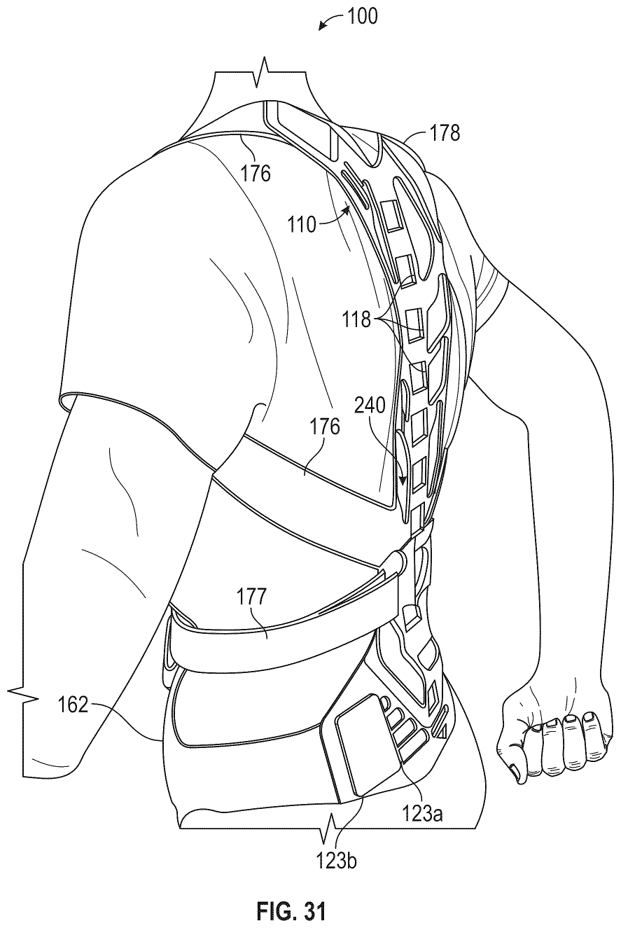

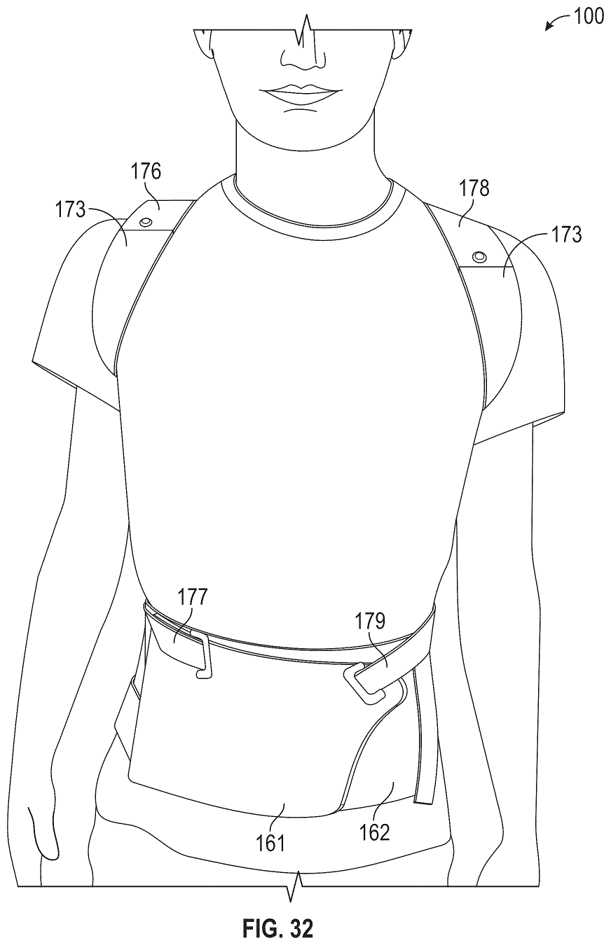

FIGS. 27-32 illustrate various example alternative embodiments of brace 100. For example, FIG. 27 illustrates bracing portion 110 paired with an alternative harness 240 that is also configured to be secured into any one of the plurality of apertures 118 in metallic stay 112 and bracing portion 110. Instead of having first and second slots 141, 142 disposed parallel to symmetrically sloping sides of the harness as with harness 140, harness 240 may comprise a base 245 and a plurality of buckles, each pivotally coupled to base 245 and comprising a respective slot 241, 242, 243, 244. A backside of harness 240 may comprise substantially similar features to harness 140, e.g., extension 143, snap clips 144 and spacers 145.

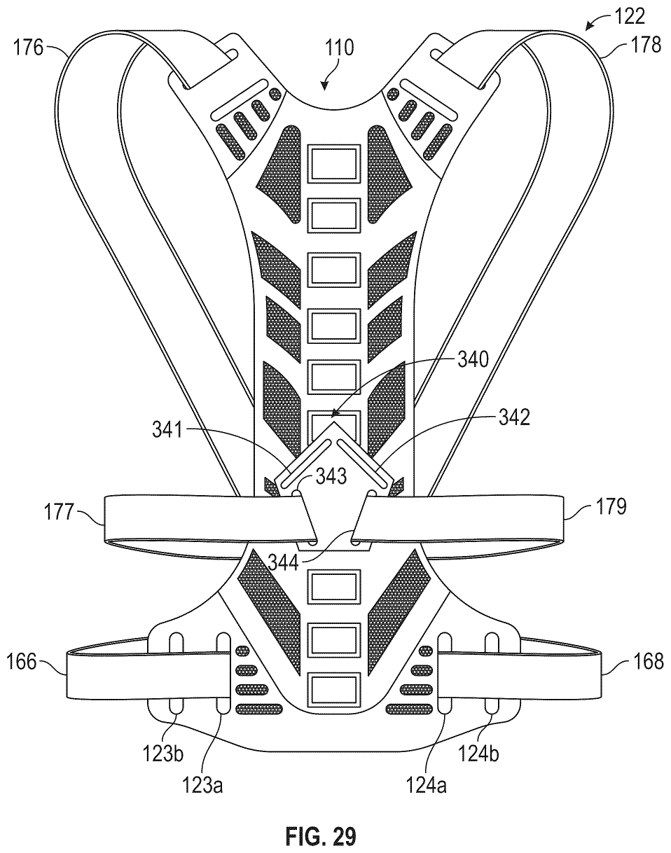

In yet another alternative, according to FIG. 29, bracing portion 110 may be paired with a harness 340 comprising a plurality of slots 341, 342, 343, 344, each disposed in a different orientation therein. Harnesses 240, 340 function in the same fashion as one another, except that, for harness 240, the buckles in which slots 241, 242, 243, 244 are disposed are configured to pivot with respect to base 245, while slots 341, 342, 343, 344 in harness 340 are not.

When bracing portion 110 is paired with either harness 240 or harness 340, different straps may be utilized. For example, while FIGS. 21A-26 use first and second straps 172, 174 and single waist strap 160, FIGS. 29-32 may functionally break first strap 172 up into a first shoulder strap 176 that couples between slot(s) 121a-b of bracing portion 110 and slot 241, 341 of harness 240, 340, and a first side strap that couples between slot 243, 343 of harness 240, 304 and slot(s) 223a-b of bracing portion 110. Likewise, FIGS. 29-32 may functionally break second strap 174 up into a second shoulder strap 177 that couples between slot(s) 122a-b of bracing portion 110 and slot 242, 342 of harness 240, 340, and a second side strap that couples between slot 244, 344 of harness 240, 304 and slot(s) 224a-b of bracing portion 110. FIGS. 29-32 may also functionally break waist second strap 160 up into a first waist strap 166 comprising one of inner front closure 161 and outer front closure 162 and a second waist strap 168 comprising the other of inner front closure 161 and outer front closure 162.

In some embodiments, a range of a posterior height of brace 100 may be from below 43 cm to about 76 cm. And an anterior height of brace 100 may be smaller than this posterior height. With respect to this disclosure, any of the aforementioned strap slots may be formed directly into the component in which the slot is described as being disposed. Alternatively, any of the aforementioned strap slots may be disposed in a buckle that is, itself, fixedly and/or pivotally coupled to the component in which the slot is described as being disposed.

Example Method(s) of Use and/or Manufacture

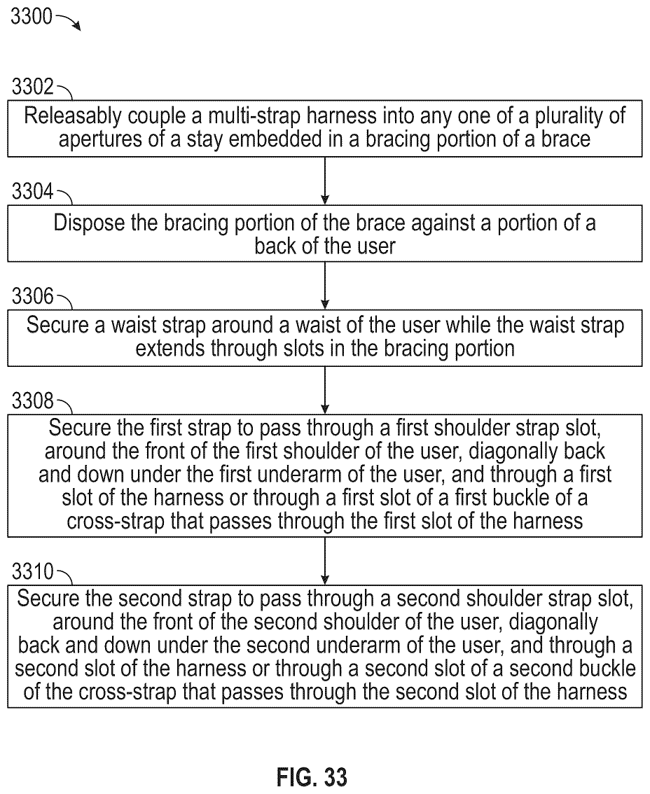

The disclosure now turns to FIG. 33, which illustrates a flowchart 3300 related to a method of utilizing a utilizing a brace, as described anywhere in this disclosure. Although particular steps are described herein, the present application is not so limited and alternative methods may include a subset of these steps, in the same or different order, and may additionally include one or more addition steps not described herein.

Step 3302 includes releasably coupling the multi-strap harness into any one of the plurality of apertures of a stay embedded in a bracing portion of the brace. For example, as previously described in connection with at least at least one FIG., multi-strap harness 140,240,340 may be releasably coupled into any one of the plurality of apertures 118 in stay 112 embedded in bracing portion 110 of brace 100.

Step 3304 includes disposing the bracing portion of the brace against a portion of the back of the user. For example, as previously described in connection with at least one FIG., bracing portion 110 of brace 100 may be disposed against a portion of the back of the user. In some embodiments, disposing the bracing portion of the brace against a portion of the back of the user may include shaping, bending or otherwise manipulating bracing portion 110 (and, e.g., metallic stay 120 embedded therein) of brace 100 to conform to the back curvature 600 of the user.

Step 3306 includes securing a waist strap around a waist of the user while the waist strap extends through slots in the bracing portion. For example, as previously described in connection with at least one FIG., waist strap 160 or first 166 and second 168 waist straps may be secured around the waist of the user while waist strap 160 or at least a portion of each of the first and second waist straps 166, 168 extend through slots 123 and 124 (or through slots 123a, 123b and 124a, 124b) of bracing portion 110.

Step 3308 includes securing a first strap to pass through a first shoulder strap slot, around the front of the first shoulder of the user, diagonally back and down under the first underarm of the user, and through a first slot of the harness or through a first slot of a first buckle of a cross-strap that passes through the first slot of the harness. For example, as previously described in connection with at least one FIG., first strap 172 or first shoulder strap 176 may be secured to pass through a first shoulder strap slot (e.g., 121, 121a or 121b), around the front of the first shoulder of the user, diagonally back and down under the first underarm of the user, and through first slot 141,241,341 of harness 140, 240, 340 or through first slot 181 of the first buckle of cross-strap 180, while cross-strap 180 passes through first slot 141,241,341 of harness 140, 240, 340.

Step 3310 includes securing a second strap to pass through a second shoulder strap slot, around the front of the second shoulder of the user, diagonally back and down under the second underarm of the user, and through a second slot of the harness or through a second slot of the second buckle of the cross-strap passing through the second slot of the harness. For example, as previously described in connection with at least one FIG., second strap 174 or second shoulder strap 178 may be secured to pass through a second shoulder strap slot (e.g., 122, 122a, or 122b), around the front of the second shoulder of the user, diagonally back and down under the second underarm of the user, and through second slot 142, 242, 342 of harness 140, 240, 340 or through second slot 182 of the second buckle of cross-strap 180, while cross-strap 180 passes through second slot 142, 242, 342 of harness 140, 240, 340.

In some such methods utilizing first strap 172, first strap 172 may be further secured to pass from first slot 141 of harness 140 or first slot 183 of the first buckle 181 of cross strap 180 diagonally down around a first side of the user to couple to an anterior portion of waist strap 160. Likewise, in such embodiments utilizing second strap 174, second strap 174 is further secured to pass from second slot 142 of harness 140 or of second buckle 183 of cross strap 180 diagonally down around the second side of the user to couple to an anterior portion of waist strap 160.

In some other embodiments, the method may also include securing the first side strap 177 to pass from third slot 243, 343 of harness 240, 340, diagonally down around the first side of the user to couple to an anterior portion of waist strap 160 or, where waist strap 160 comprises first and second straps 166, 168, to an anterior portion of first waist strap 166. Likewise, in such embodiments, the method also includes securing second side strap 179 to pass from fourth slot 244, 344 of harness 240,340 diagonally down around the second side of the user to couple to an anterior portion of waist strap 160 or, where waist strap 160 comprises first and second straps 166, 168, an anterior portion of second waist strap 168.

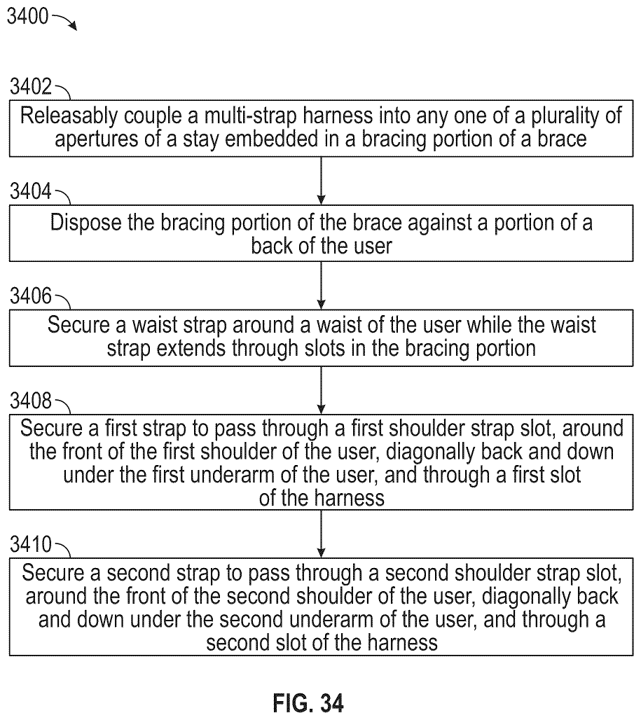

The disclosure now turns to FIG. 34, which illustrates a flowchart 3400 related to another method of utilizing a utilizing a brace, as described anywhere in this disclosure. Although particular steps are described herein, the present application is not so limited and alternative methods may include a subset of these steps, in the same or different order, and may additionally include one or more addition steps not described herein.

Step 3402 includes releasably coupling a multi-strap harness into any one of the plurality of apertures of a stay embedded in a bracing portion of a brace. For example, as previously described in connection with at least at least one FIG., multi-strap harness 140 may be releasably coupled into any one of the plurality of apertures 118 of stay 112, which is embedded in bracing portion 110 of brace 100.

Step 3404 includes disposing the bracing portion of the brace against a portion of the back of the user. For example, as previously described in connection with at least one FIG., bracing portion 110 of brace 100 may be disposed against a portion of the back of the user. In some embodiments, disposing the bracing portion of the brace against a portion of the back of the user may include shaping, bending or otherwise manipulating bracing portion 110 (and, e.g., metallic stay 120 embedded therein) of brace 100 to conform to the back curvature 600 of the user.

Step 3406 includes securing a waist strap around the waist of the user while the waist strap extends through slots of the bracing portion. For example, as previously described in connection with at least one FIG., waist strap 160 may be secured around the waist of the user while waist strap 160 extends through slots 123 and 124 (or through one or both of slots 123a, 123b and one or both of 124a, 124b) of bracing portion 110.

Step 3408 includes securing a first strap to pass through a first shoulder strap slot, around the front of the first shoulder of the user, diagonally back and down under the first underarm of the user, and through a first slot of the harness. For example, as previously described in connection with at least one FIG., first strap 172 may be secured to pass through first shoulder strap slot 121 (or one or both of slots 121a, 121b), around the front of the first shoulder of the user, diagonally back and down under the first underarm of the user, and through first slot 141 of harness 140.

Step 3410 includes securing a second strap to pass through a second shoulder strap slot, around the front of the second shoulder of the user, diagonally back and down under the second underarm of the user, and through a second slot of the harness. For example, as previously described in connection with at least one FIG., second strap 174 may be secured to pass through second shoulder strap slot 122 (or one or both of slots 122a, 122b), around the front of the second shoulder of the user, diagonally back and down under the second underarm of the user, and through second slot 142 of harness 140.

In some such methods, first strap 172 may be further secured to pass from first slot 141 of harness 140 diagonally down around a first side of the user to couple to an anterior portion of waist strap 160. Likewise, in such embodiments, second strap 174 is further secured to pass from second slot 142 of harness 140 diagonally down around the second side of the user to couple to an anterior portion of waist strap 160.

A method of manufacturing a brace, as described anywhere in this disclosure is also contemplated. Such a method may include providing, securing, forming, molding, bending, stamping, casting, printing, assembling and/or otherwise preparing any feature or element of a brace, as described anywhere in this disclosure.

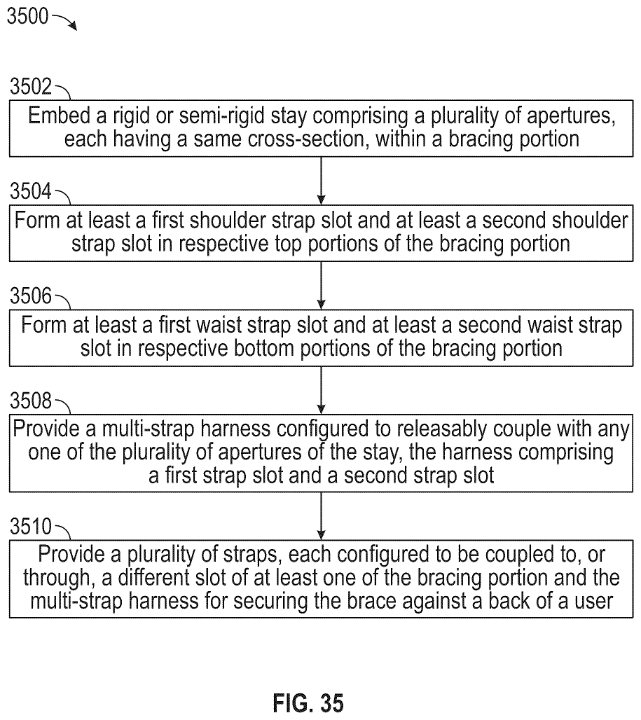

An example of such a method is illustrated in FIG. 35 and flowchart 3500. Although particular steps are described herein, the present application is not so limited and alternative methods may include a subset of these steps, in the same or different order, and may additionally include one or more addition steps not described herein.

Step 3502 includes embedding a rigid or semi-rigid stay comprising a plurality of apertures, each having a same cross-section, within a bracing portion. For example, as previously described in connection with at least at least one FIG., rigid or semi-rigid stay 112, comprising a plurality of apertures 118, each having a same cross-section, may be embedded within bracing portion 110 as, or to include material layers 120 as described anywhere in this disclosure.

Step 3504 includes forming at least a first shoulder strap slot and at least a second shoulder strap slot in respective top portions of the bracing portion. For example, as previously described in connection with at least at least one FIG., at least a first shoulder strap slot 121, 121a, 121b and at least a second shoulder strap slot 122, 122a, 122b may be formed in respective top portions of bracing portion 100, as described anywhere in this disclosure.

Step 3506 includes forming at least a first waist strap slot 123, 123a, 123b and at least a second waist strap slot 124, 124a, 124b in respective bottom portions of the bracing portion. For example, as previously described in connection with at least at least one FIG., at least a first waist strap slot 123, 123a, 123b and at least a second waist strap slot 124, 124a, 124b may be formed in respective bottom portions of bracing portion 110.

Step 3508 includes providing a multi-strap harness configured to releasably couple with any one of the plurality of apertures of the stay, the harness comprising a first strap slot and a second strap slot. For example, as previously described in connection with at least one FIG., multi-strap harness 140, comprising a first strap slot 141 and a second strap slot 142 and configured to releasably couple with any one of the plurality of apertures 118 of stay 112, may be provided.

Step 3510 includes providing a plurality of straps, each configured to be coupled to, or through, a different slot of at least one of the bracing portion and the multi-strap harness for securing the brace against a back of a user. For example, as previously described in connection with at least at least one FIG., a plurality of straps 160, 172, 174 may be provided, each configured to be coupled to, or through, a different slot of at least one of the bracing portion and the multi-strap harness for securing the brace against a back of a user.

In some such methods, stay 112 is formed such that a back-facing side of central portion 114 of stay 112, extending from a top to a bottom of stay 112, comprises a concave curvature such that central portion 114 extends away from lateral portions 115 of stay 112 that extend from the top to the bottom of stay 112 along each side of central portion 115.

In some such methods, embedding the stay within the bracing portion comprises at least one of disposing textile layer 152 against a first side of stay 112, disposing outer layer 151, comprising polyurethane, against an outer surface of textile layer 152, disposing padding layer 153 against a second side of stay 112 and against textile layer 152, and disposing inner textile layer 154 against an inner surface of padding layer 153.

In some such methods, harness 140 is formed to have a substantially trapezoidal shape, a top edge of the harness being wider than a parallel bottom edge of the harness and opposing side edges symmetrically sloping between the top edge and the bottom edge.

In some such methods, a backside of the harness is formed to comprise extension 143 having one or more clips 144 configured to interface with any one of apertures 118 in stay 112 of bracing portion 110.

In some such methods, the backside of harness 140 is formed to comprise one or more spacers 145 configured to contact bracing portion 110 when harness 140 is properly disposed in one of apertures 118.

In some such methods, stay 112 is configured to be manipulated to have a dorsal curvature in the sagittal plane that matches a dorsal curvature of a back of a user. In some such methods, a back-facing side of upper portion 111 of stay 112 comprises a concave dorsal curvature that substantially mimics a dorsal curvature of a lower thoracic portion of the back of the user. In some such methods, a back-facing side of lower portion 113 of stay 112 comprises a convex dorsal curvature that substantially mimics a dorsal curvature of a lumbar portion of the back of the user. In some such methods, stay 112 is formed such that upper portion 111 has a first width and lower portion 113 of stay 112 flares from the first width to a second width greater than the first width. In some such methods, stay 112 is formed such that a back-facing side of central portion 114, extending from a top to a bottom of stay 112, comprises a concave curvature such that central portion 114 extends away from lateral portions 115 of stay 112 that extend from the top to the bottom of stay 112 along each side of central portion 114.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

What is claimed is:

1. A back brace, comprising:
a bracing portion configured to be disposed against a portion of a back of a user, the bracing portion comprising:
a rigid or semi-rigid stay comprising a plurality of apertures, each having a same cross-section,
at least a first shoulder strap slot and at least a second shoulder strap slot, each disposed in a respective top portion of the bracing portion, and
at least a first waist strap slot and at least a second waist strap slot, each disposed in a respective bottom portion of the bracing portion;
a multi-strap harness configured to releasably couple with any one of the plurality of apertures of the stay, the harness comprising a first strap slot and a second strap slot; and
a plurality of straps, each coupled to a different slot of at least one of the bracing portion and the multi-strap harness to, thereby, wrap at least partly around a waist, a respective side and/or a respective shoulder of a user.

2. The brace of claim 1, wherein the stay comprises metal.

3. The brace of claim 1, wherein the stay comprises a dorsal curvature in the sagittal plane configured to match a dorsal curvature of the back of the user.

4. The brace of claim 3, wherein a back-facing side of an upper portion of the stay comprises a concave dorsal curvature that substantially mimics a dorsal curvature of a lower thoracic portion of the back of the user.

5. The brace of claim 3, wherein a back-facing side of a lower portion of the stay comprises a convex dorsal curvature that substantially mimics a dorsal curvature of a lumbar portion of the back of the user.

6. The brace of claim 1, wherein an upper portion of the stay has a first width and a lower portion of the stay flares from the first width to a second width greater than the first width.

7. The brace of claim 1, wherein a back-facing side of a central portion of the stay, extending from a top to a bottom of the stay, comprises a concave curvature such that the central portion extends away from lateral portions of the stay that extend from the top to the bottom of the stay along each side of the central portion.

8. The brace of claim 7, wherein the apertures are disposed within the central portion of the stay.

9. The brace of claim 1, wherein the bracing portion comprises an outer layer comprising polyurethane.

10. The brace of claim 9, wherein the bracing portion comprises a textile layer disposed against the outer layer.

11. The brace of claim 10, wherein the bracing portion comprises a padding layer disposed against the textile layer and against the stay.

12. The brace of claim 11, wherein the bracing portion comprises an inner textile layer.

13. The brace of claim 1, wherein the harness has a substantially trapezoidal shape, a top edge of the harness being wider than a parallel bottom edge of the harness and opposing side edges symmetrically sloping between the top edge and the bottom edge.

14. The brace of claim 13, wherein a backside of the harness comprises an extension having one or more clips configured to interface with any one of the plurality of apertures in the stay of the bracing portion.

15. The brace of claim 14, wherein the backside of the harness comprises one or more spacers configured to contact the bracing portion when the harness is properly disposed in one of the plurality of apertures, thereby stabilizing the harness within the aperture.

16. The brace of claim 1, wherein the plurality of straps comprises a waist strap, comprising:
a central strap portion configured to be threaded through the first and second waist strap slots of the bracing portion,
an inner front closure coupled to one end of the central strap portion, and
an outer front closure coupled to another end of the central strap portion.

17. The brace of claim 1, wherein the plurality of straps comprises a first strap configured to pass through the first shoulder strap slot, around a front of a first shoulder of the user, diagonally back and down under a first underarm of the user, through the first slot of the harness, diagonally down around a first side of the user to couple to an anterior portion of the waist strap.

18. The brace of claim 17, wherein the plurality of straps comprises a second strap configured to pass through the second shoulder strap slot, around a front of a second shoulder of the user, diagonally back and down under a second underarm of the user, through the second slot of the harness, diagonally down around a second side of the user to couple to an anterior portion of the waist strap.

19. A method of utilizing a back brace, the method comprising:
releasably coupling a multi-strap harness into any one of a plurality of apertures of a rigid or semi-rigid stay embedded in a bracing portion of the brace;
disposing the bracing portion of the brace against a portion of the back of the user;
securing a waist strap around a waist of the user while the waist strap extends through slots in the bracing portion;
securing a first strap to pass through a first shoulder strap slot of the bracing portion, around the front of the first shoulder of the user, diagonally back and down under the first underarm of the user, and through a first slot of the harness; and
securing a second strap to pass through a second shoulder strap slot of the bracing portion, around the front of the second shoulder of the user, diagonally back and down under the second underarm of the user, and through a second slot of the harness.

20. A method of manufacturing a brace, the method comprising:
embedding a rigid or semi-rigid stay comprising a plurality of apertures, each having a same cross-section, within a bracing portion;
forming at least a first shoulder strap slot and at least a second shoulder strap slot in respective top portions of the bracing portion;
forming at least a first waist strap slot and at least a second waist strap slot in respective bottom portions of the bracing portion;
providing a multi-strap harness configured to releasably couple with any one of the plurality of apertures of the stay, the harness comprising a first strap slot and a second strap slot; and
providing a plurality of straps, each configured to be coupled to, or through, a different slot of at least one of the bracing portion and the multi-strap harness for securing the brace against a back of a user.
